(19) United States
(12) Patent Application Publication
(10) Pub. No.: US 2018/0085151 A1
(43) Pub. Date: Mar. 29, 2018

(54) Ankle (Tibio-Talar) Fusion Nail

(71) Applicant: Texas Tech University System, Lubbock, TX (US)

(72) Inventor: Amr Abdelgawad, El Paso, TX (US)

(21) Appl. No.: 15/566,280

(22) PCT Filed: Apr. 14, 2016
(86) PCT No.: PCT/US2016/027455

§ 371 Date: Oct. 13, 2017

Related U.S. Application Data
(60) Provisional application No. 62/148,277, filed on Apr. 16, 2015.

(51) Int. Cl. A61B 17/72; A61B 17/84

(52) U.S. Cl. CPC A61B 17/7291; A61B 17/846; A61B 17/7241

(57) ABSTRACT

The present invention includes a tibio-talar device and a method for providing stabilizing support between a tibia and a talus comprising: a bone nail adapted to traverse the talus and the tibia, wherein the bone nail is curved such that it traverses the talus and enters the tibia at an angle, wherein the nail is angled from side to side, wherein the bone nail is configured to provide for intra medullary fusion of the ankle without obstructing the subtalar joint. The nail is introduced from talus and curves to pass to the tibia fusing the ankle joint.

### TECHNICAL FIELD OF THE INVENTION

The present invention relates in general to the field of arthrodesis, and more particularly, to a novel ankle (tibio-talar) fusion nail.

### BACKGROUND OF THE INVENTION

Without limiting the scope of the invention, its background is described in connection with Joint arthrodesis and arthroplasty.

Ankle fusion is currently done by screws with or without plates or external fixators. Both of these have their disadvantages. The nail system when used for nail fixation requires fixation of the subtalar joint. Thus, when a nail is used to fuse an ankle (tibio-talar) joint, it will have to also fuse another joint (subtalar or the talo-calcaneal joint). This means that the ankle joint cannot be fused with a nail unless another joint is fused with it.

U.S. Pat. No. 8,585,744, issued to Duggal, et al., for a Joint arthrodesis and arthroplasty. Briefly, these inventors teach an implantable fixation system for fusing a joint between a first bone and a second bone. The system may include an anchor, standoff, bolt, and cortical washer. The system may be implanted across the joint along a single trajectory, the length of the system adjustable to provide compressive force between the anchor and the cortical washer. The system may be implanted across a tibiotalar joint with the anchor positioned in the sinus tarsi. A spacing member may be inserted between the two bones and the fixation system implanted to extend through an opening in the spacing member. The spacing member may be anatomically shaped and/or provide deformity correction. An ankle arthroplasty system may include a tibial plate, a talar plate, and a bearing insert. The plates may be anchored to the tibia and talus along a single trajectory. The ankle arthroplasty system may be revisable to a fusion system.

### SUMMARY OF THE INVENTION

In one embodiment, the present invention includes a tibio-talar device for providing stabilizing support between a tibia and a talus comprising: a bone nail adapted to traverse the tibia and the talus, wherein the bone nail is curved such that it traverses the tibia and enters the talus at an angle, wherein the nail is angled from lateral to medial and anterior to posterior, wherein the bone nail is configured to provide for intra medullary fusion of the ankle without obstructing the subtalar joint. In one aspect, the nail is further affixed to the tibia and talus with a biocompatible adhesive. In another aspect, the nail is contoured to lock proximally in the tibia from lateral to medial. In another aspect, the bone nail is provided in increments of approximately 5 cm. In another aspect, the bone nail is at least one of titanium or stainless steel. In another aspect, the bone nail has an upper and a lower portion, wherein the upper portion and the lower portion have the same curvature, the upper portion is straight and the lower portion is curved, or wherein the lower and upper portion are curved but have a radius of curvature. In another aspect, the bone nail has 2, 3, 4, 5, 6, or 8 openings that each are capable of supporting a screw, wherein the holes are positioned along the length of the bone nail, and wherein optionally one of the holes permits affixing the bone screw to the talus.

In another embodiment, the present invention includes a ankle arthrodesis nail comprising: a bone nail adapted to traverse the tibia and the talus, wherein the bone nail is curved such that it traverses the tibia and enters the talus at an angle, wherein the nail is angled from lateral to medial and anterior to posterior, and wherein the bone nail is configured to provide for intra medullary fusion of the ankle, wherein the nail locks proximally in the tibia from lateral to medial, without obstructing the subtalar joint. In one aspect, the nail is further affixed to the tibia and talus with a biocompatible adhesive. In another aspect, the nail is contoured to lock proximally in the tibia from lateral to medial. In another aspect, the bone nail is provided in increments of approximately 5 cm. In another aspect, the bone nail is at least one of titanium or stainless steel. In another aspect, device further comprises one or more opening to receive screws that pass from the tibia or the fibula through the nail to the talus. In another aspect, the bone nail has an upper and a lower portion, wherein the upper portion and the lower portion have the same curvature, the upper portion is straight and the lower portion is curved, or wherein the lower and upper portion are curved but have a radius of curvature. In another aspect, the bone nail has 2, 3, 4, 5, 6, or 8 openings that each are capable of supporting a screw, wherein the holes are positioned along the length of the bone nail, and wherein optionally one of the holes permits affixing the bone screw to the talus.

In yet another embodiment, the present invention includes a method for conducting an ankle arthrodesis system for providing stabilizing support between a tibia and a talus, the system comprising: identifying a patient in need of an ankle arthrodesis; a bone nail adapted to traverse the tibia and the talus, wherein the bone nail is curved such that it traverses the tibia and enters the talus at an angle, wherein the nail is angled from lateral to medial and anterior to posterior, wherein the bone nail is configured to provide for intra medullary fusion of the ankle; and anchoring the bone nail without obstructing the subtalar joint. In one aspect, the opening is defined further as being adapted for the bone anchor to at least one of traverse from talus to tibia or traverse from tibia to talus. In another aspect, the nail is further affixed to the tibia and talus with a biocompatible adhesive. In another aspect, the nail is contoured to lock proximally in the tibia from lateral to medial. In another aspect, the bone nail is provided in increments of approximately 5 cm. In another aspect, the bone nail is at least one of titanium or stainless steel. In another aspect, the method further comprising inserting one or more screws through openings in the bone nail, wherein the screws pass from the tibia or the fibula through the nail to the talus. In another aspect, the bone nail has an upper and a lower portion, wherein the upper portion and the lower portion have the same curvature, the upper portion is straight and the lower portion is curved, or wherein the lower and upper portion are curved but have a radius of curvature. In another aspect, the bone nail has 2, 3, 4, 5, 6, or 8 openings that each are capable of supporting a screw, wherein the holes are positioned along the length of the bone nail, and wherein optionally one of the holes permits affixing the bone screw to the talus.

### DESCRIPTION OF THE DRAWINGS

For a more complete understanding of the features and advantages of the present invention, reference is now made to the detailed description of the invention along with the accompanying figures and in which:

FIG. 1 shows a lateral and an anterior view of the anatomy of the ankle and subtalar joint.

FIG. 2 shows a posterior view of an ankle fusion by plate of the prior art.

FIG. 3 shows an anterior view of an ankle fusion by screws of the prior art.

FIG. 4 shows an isometric view of an ankle fusion by external fixator of the prior art.

FIG. 5 shows a lateral view of an ankle fusion by nail of the prior art (which will lead to subtalar fusion).

FIG. 6 shows a lateral and a posterior view of an ankle fusion by nail of the prior art (which will lead to subtalar fusion).

FIG. 7 shows an isometric anterior view of the new device positioned between the tibia and the talus.

FIG. 8 shows a close-up lateral view of the new device positioned between the tibia and the talus (as seen from the anterior lateral view).

FIG. 9 shows an anterior view of the new device positioned between the tibia and the talus on a MRI scan.

FIG. 10 shows a close-up lateral view of the new device positioned between the tibia and the talus on a MRI scan.

FIG. 11 shows a coronal cut of a CT showing the new device positioned between the tibia and the talus.

FIG. 12 shows a computational analysis was performed applying an axial load of 350N at the top area of the tibia simulating the weight bearing of an average person.

FIG. 13 shows a computational analysis in which the talus is subjected to high stress in the section around the nail insertion.

FIG. 14 shows a computational analysis in which the nail, tibia, and talus are shown and in which the stress is concentrated in the upper section, which carries the axial load at the first instance.

FIG. 15 shows the fixation of the nail to the bones with screws that ensures proper fixation and preventing movement during joint fusion and supporting and distributing stresses uniformly throughout the structure.

FIG. 16 shows the same image as in FIG. 15, but illustrates screw 5. Screw 5 is inserted in an inclined configuration, which is positioned from the tibia to the talus to increase fixation.

FIG. 17 shows the screw of the talus (4) and the screw which crosses from tibia to talus (5) have angle of inclination.

FIGS. 18A and 18B illustrate the angle of inclination for talus screw (4) and for tibia/talus screw (5).

FIGS. 19A and 19B illustrate one option for the nail of the present invention in which two straight segments connected each other; they have an angle of 37.24°. This nail is called “straight nail” and it is inserted from the talus to the tibia.

FIG. 19C shows that the subtalar joint is not affected when the nail is inserted and positioned. The image below shows the clearance between the nail and calcaneus or between the nail and subtalar joint.

FIGS. 20A and 20B are a lateral and front view of the nail and screw holes. FIG. 20C is an isometric view of the nail, and screws in the screw holes.

FIGS. 21A and 21B illustrate another option for the nail of the present invention in which two straight segments connected each other and shows the clearance between the nail and calcaneus or between the nail and subtalar joint, and is based on constant curved nail geometry. Only one curvature is designed and will pass through the tibia and the talus.

FIG. 21C shows the clearance between the nail trajectory and the subtalar joint is shown below; it seems to be good enough to prevent damage in the subtalar joint. In this embodiment, the nail is not as close to the calcaneus as in the figures above, however the nail is closer to the sidewall of the tibia.

FIGS. 22A and 22B are a lateral and front view of the nail and screw holes. FIG. 22C is an isometric view of the nail, and screws, in the screw holes.

FIGS. 23A and 23B illustrate another option for the nail of the present invention in which the curved nail has two different curves connected to form the nail. One curvature is designed for the tibia segment and a more pronounce curvature is selected for the talus segment.

FIG. 23C shows the clearance between the nail and subtalar joint is small but still enough to avoid damage in the calcaneus zone.

FIGS. 24A and 24B are a lateral and front view of the nail and screw holes. FIG. 24C is an isometric view of the nail, screws in the screw holes.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Ankle fusion is currently done by screws, plus or minus, plates or external fixator. Both of these have their disadvantages. The nail system when used for nail fixation has also to include the subtalar joint. So when a nail is used to fuse an ankle (tibio-talar) joint, it will have to also fuse another joint (subtalar or the talo-calcaneal joint). This means that the ankle joint cannot be fused by a nail unless another joint is fused with it. This invention to have a nail that can fuse the ankle without the need for fusing another joint with it.

Thus, the present invention provides for a novel ankle fusion nail that overcomes the problems with existing external fixators or plates, as outlined hereinbelow. Ankle fusion by current nails will require fusion of the subtalar joint. This invention helps to fuse the ankle joint with a nail without fusing the subtalar joint.

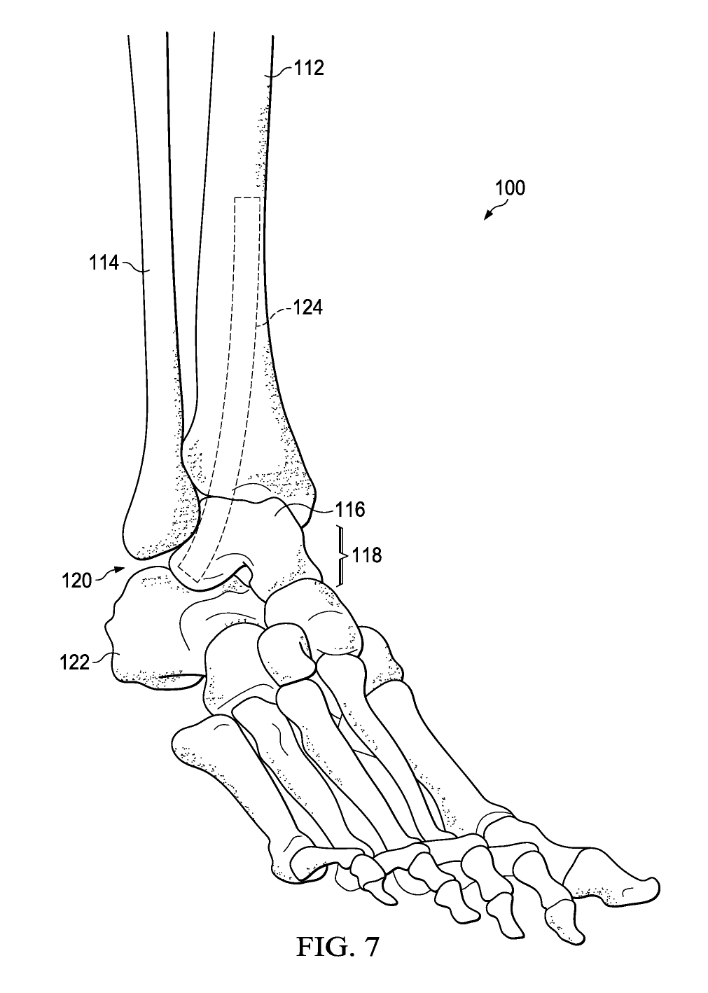

Thus, in one embodiment the present invention includes a nail that is adapted to fuse the ankle joint without crossing the subtalar joint (see FIGS. 7-11).

In certain non-limiting examples, the nail specification can include: Titanium or stainless steel, or a material of similar strength that is compatible. While not a limitation of the present invention, it may be convenient to provide the nail in a few widths, e.g., 8 mm and 10 mm (more sizes can be added). Likewise, the length can be selected from, e.g., 20-35 cm with 5 cm increments, depending on the size of the bones to be fused (e.g., pediatric versus adult). One feature of the nail is that it permits distal locking in the talus (from lateral to medial and from anterior to posterior). Another feature is that it provides for locking proximally in the tibia that is from lateral to medial. Another feature is also screws that pass from the tibia or the fibula through the nail to the talus. This will give a great amount of fixation and stability for the nail fusion construct.

Additional novel features of the present invention are that it provides for intra medullary fusion of the ankle without crossing the subtalar joint irrespective of: (1) Nail is passing from talus to tibia or from tibia to talus; (2) Shape of nail, diameter, material; (3) Size and length; and (4) orientation and number of locking screws.

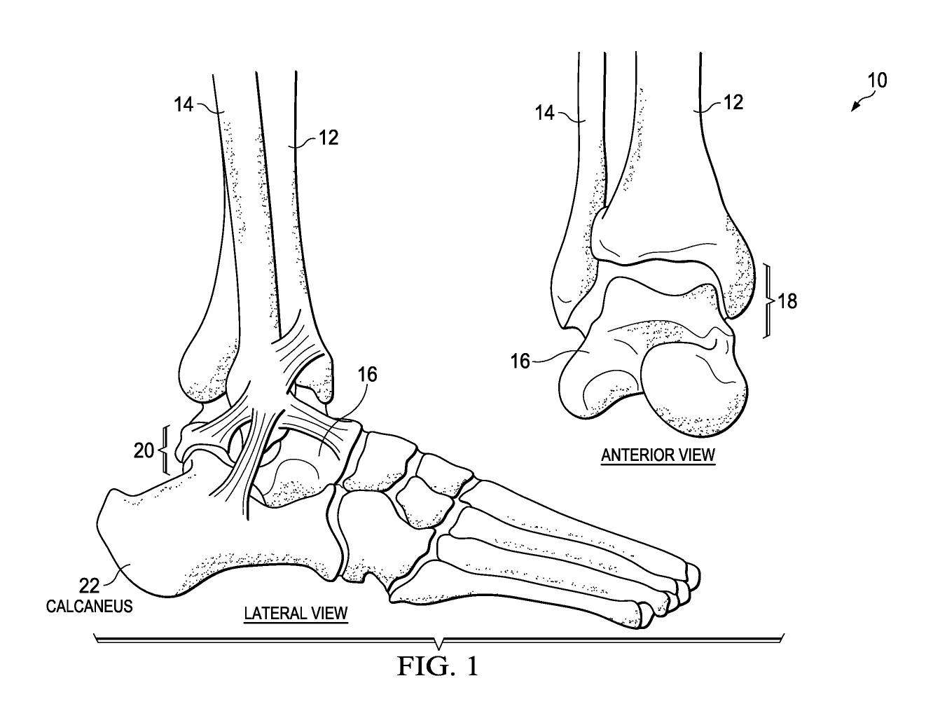

FIG. 1 shows a lateral and an anterior view of the anatomy of the ankle and subtalar joint 10. Briefly, the tibia 12 is shown in relation to fibula 14. The talus 16 is shown in both views and in relation to the true ankle joint 18 and the subtalar joint 20. Finally, in the lateral view, the calcaneus 22 is also depicted.

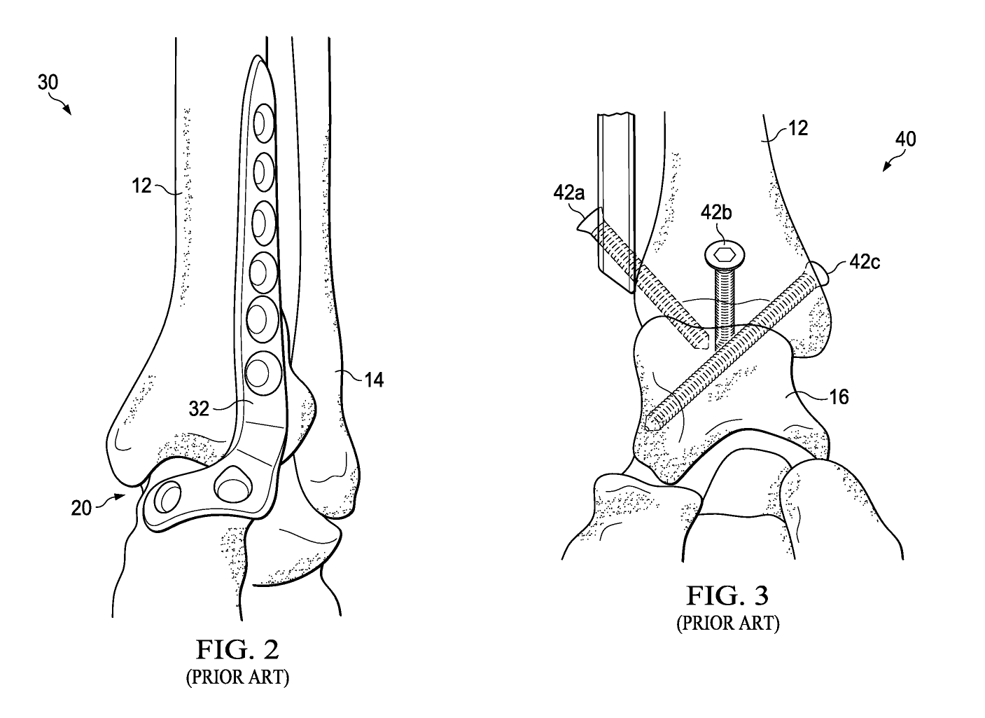

FIG. 2 shows a posterior view of an ankle fusion 30 using a plate 32 of the prior art. In this posterior view, the plate 32 is depicted connecting the tibia 12 across the ankle joint 18.

FIG. 3 shows an anterior view of an ankle fusion 40 by screws 42a, 42b and 42c depicted diagonally across the tibia 12 into the talus 16 of the prior art.

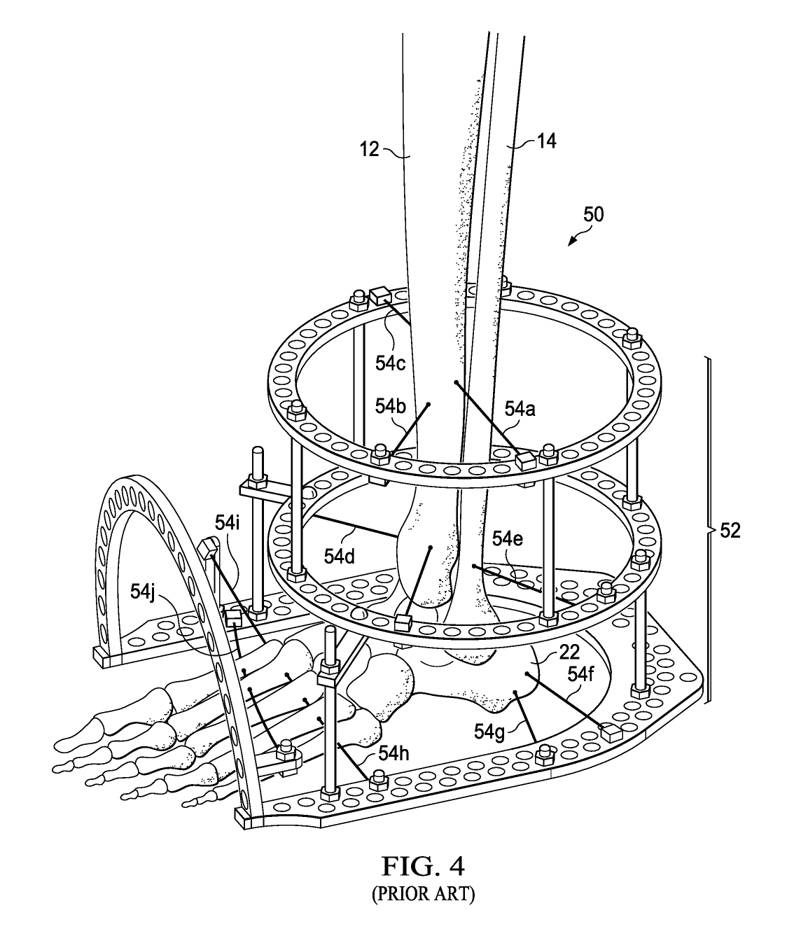

FIG. 4 shows an isometric view of an ankle fusion 50 by external fixator 52 of the prior art, in which pins 54a-j are shown connecting to the tibia 12, fibula 14, the calcaneus 22.

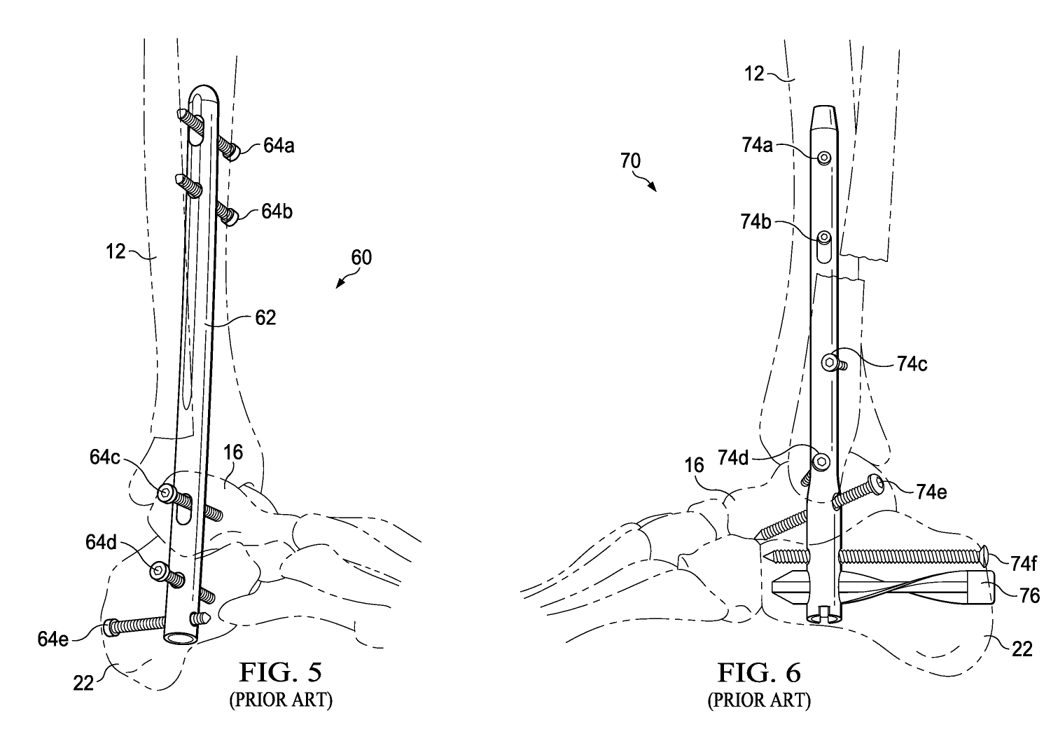

FIG. 5 shows a lateral view of an ankle fusion 60 by nail of the prior art. A straight nail 62 that is positioned inside the tibia 12 is depicted into which screws 64a-e are shown screwed into the tibia 12, talus 16 and calcaneus 22.

FIG. 6 shows a lateral and a posterior view of an ankle fusion 70 by nail of the prior art. In this version, the straight nail 72 is also depicted with various screws 74a-f and peg 76 connecting the tibia 12, the talus 16 and the calcaneus 22. In this version, the screws 74a-e are connected to nail 72, while screw 74f is screwed into the nail 72, adjacent peg 76, both of which are screwed through the calcaneus 22. Screw 74e is screwed through the subtalar joint 20.

FIG. 7 shows an isometric anterior view of an ankle fusion 100 of the present invention depicting the tibia 112 and the talus 116. Briefly, the tibia 112 is shown in relation to fibula 114. The talus 116 is shown in both views and in relation to the true ankle joint 118 and the subtalar joint 120. Finally, in the lateral view, the calcaneus 122 is also depicted. The bone nail 124 of the present invention is shown traversing the tibia 112 and connecting into the talus 116, in a manner that does not traverse the subtalar joint 120. By providing a direct connection between the tibia 112 and the talus 116, the bone nail 124 can be used alone, without the need for screws. In some embodiments (not depicted), additional mechanical support can be provided between the bones and the nail, with support found on either the nail or the bone. This will be achieved by using locking screws in both the tibia and the talus. Compression across the fusion can be obtained by having screws that pass from the tibia or fibula on one side and the talus on the other side passing though the plate and giving extra strength to the fusion construct. The curvature of the bone nail 124 permits the nail to traverse the tibia 112 and the talus 116. Because the bone nail 124 is curved, such that it traverses the talus and enters the tibia at an angle, the bone nail 124 enters at an angle that is from lateral to medial. As such, the bone nail 124 is configured to provide for intra medullary fusion of the ankle without obstructing the subtalar joint 120.

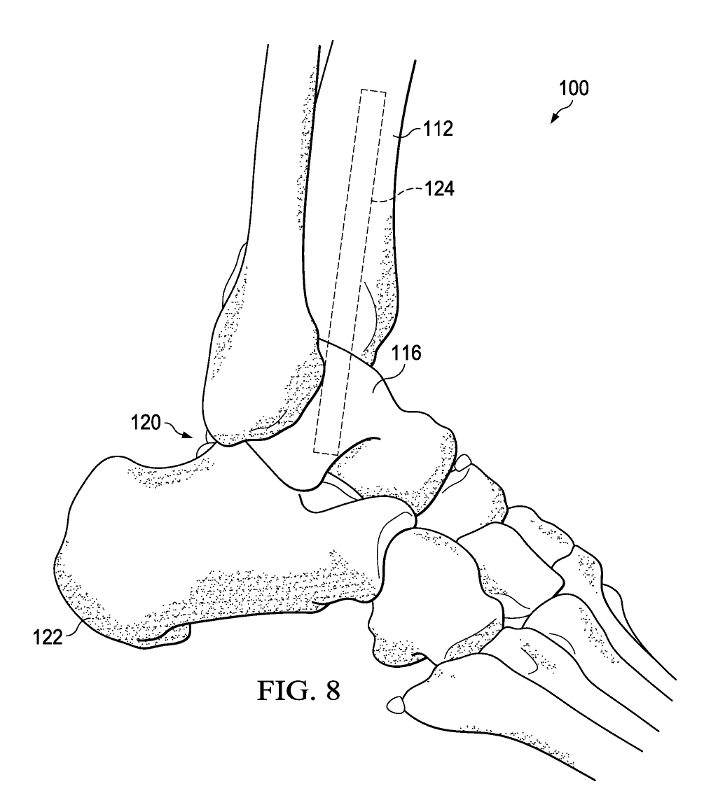

FIG. 8 shows a close-up lateral view of the bone nail 124 of the present invention positioned showing the tibia 112 and the talus 116, which in the lateral view shows that in this angle the bone nail 124 is generally straight and does not obstruct the subtalar joint 120.

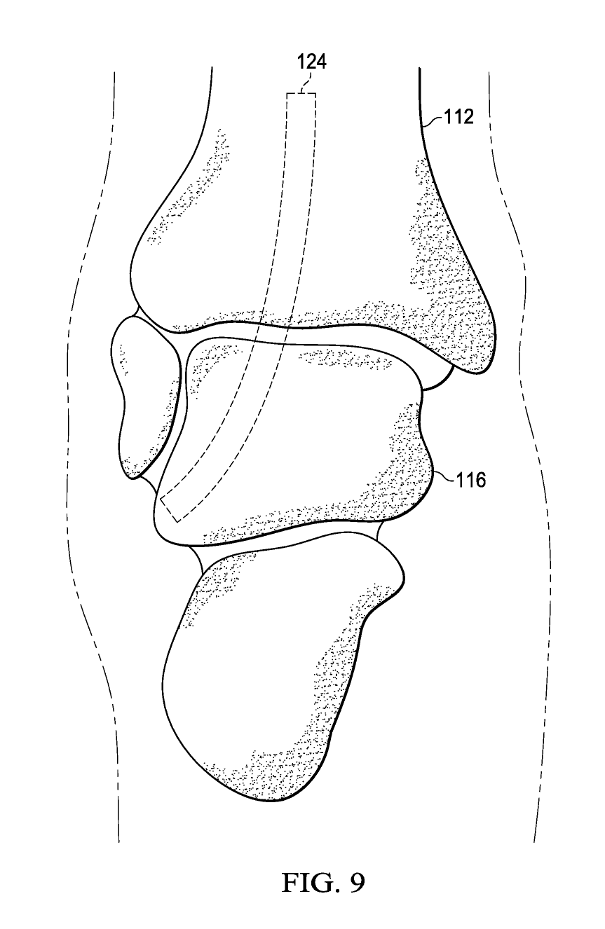

FIG. 9 shows an anterior view of the bone nail 124 of the present invention positioned showing the tibia 112 and the talus 116 superimposed on a MRI scan.

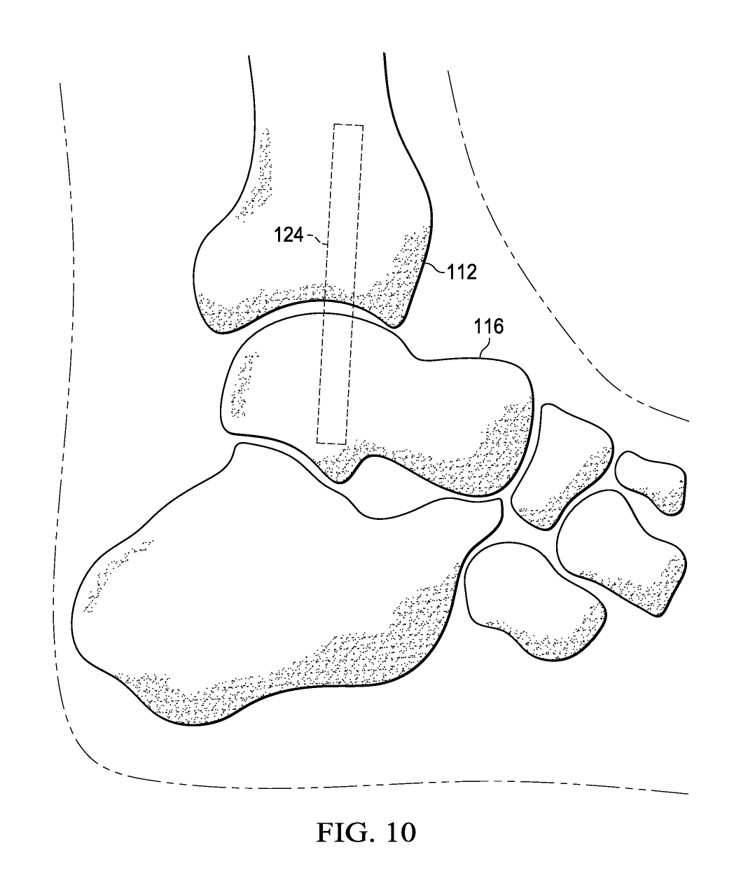

FIG. 10 shows a close-up lateral view of the bone nail 124 of the present invention positioned showing the tibia 112 and the talus 116 superimposed on a MRI scan.

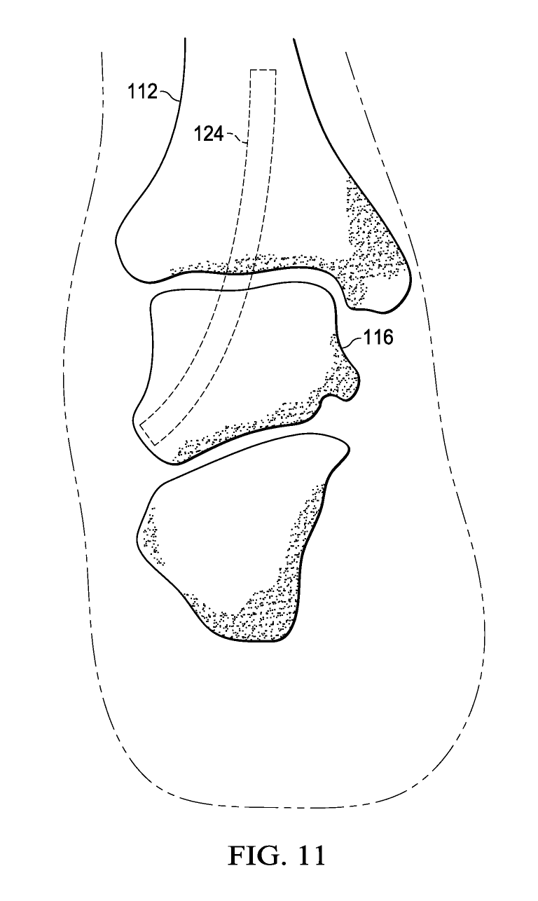

FIG. 11 shows a coronal view of the bone nail 124 of the present invention positioned showing the tibia 112 and the talus 116 superimposed on a CT scan.

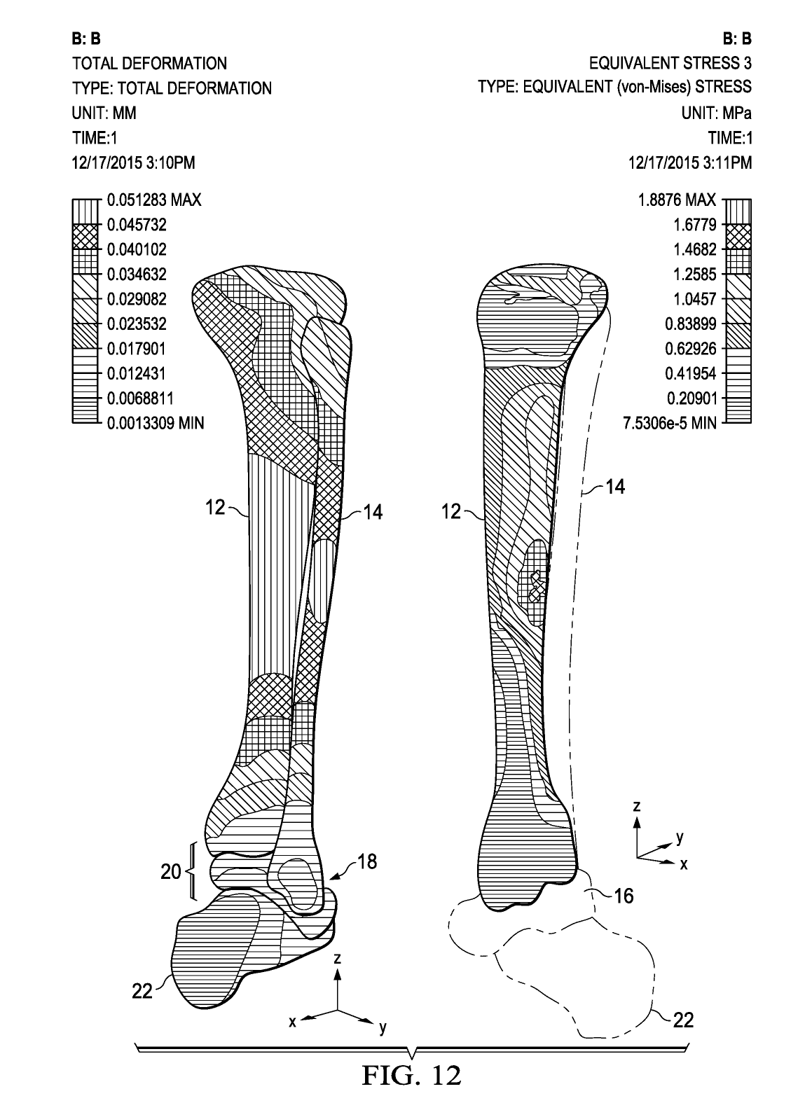

FIG. 12 shows a computational analysis was performed applying an axial load of 350N at the top area of the tibia simulating the weight bearing of an average person. Briefly, the tibia 12 is shown in relation to fibula 14. The talus 16 is shown in both views and in relation to the true ankle joint 18 and the subtalar joint 20. Finally, in the lateral view, the calcaneus 22 is also depicted. The inventors determined which the main stressed areas are in the bone and to define the most stressed sections of the nail. For this study, the nail is curved since it is expected to be the worst case if compare to straight segments carrying load, however the nail is lacking of details intentionally, it is not the final design and does not include screw holes yet, reducing the complexity of the model. For the tibia and fibula, the main and higher stresses are located close to the middle section of the bone when applying the axial force.

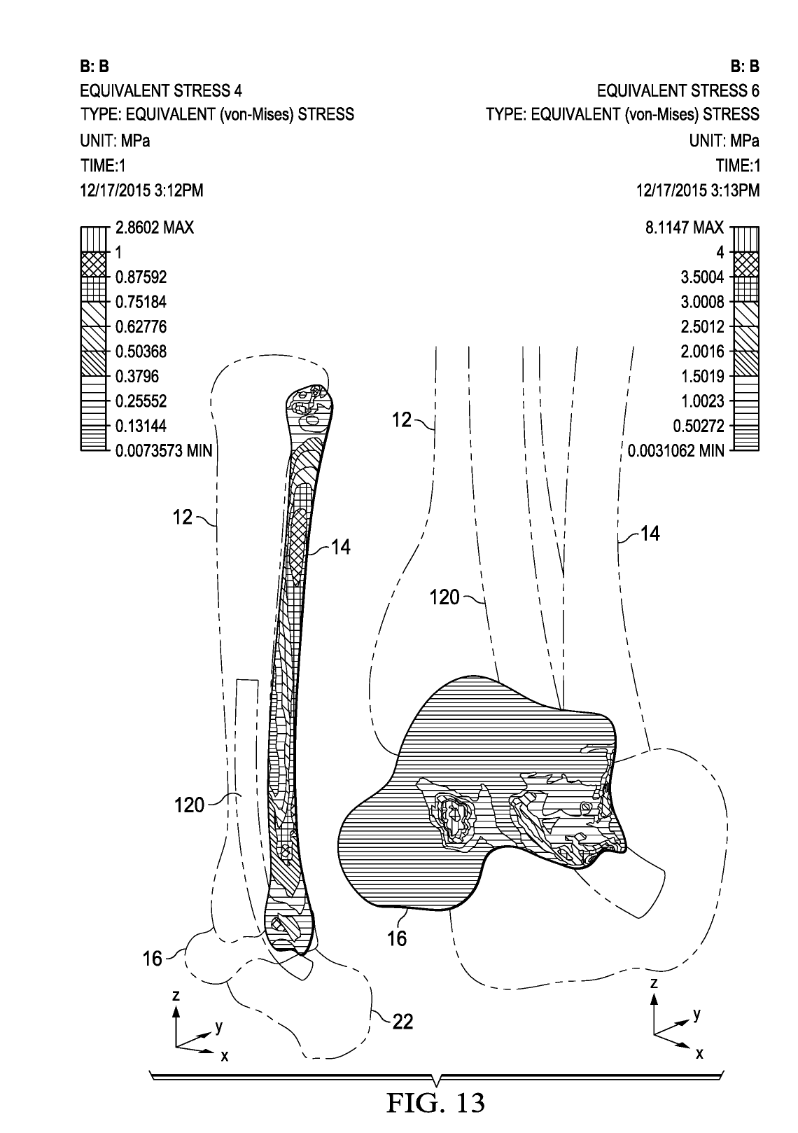

FIG. 13 shows a computational analysis in which the talus is subjected to high stress in the section around the nail insertion. The tibia 12 is shown in relation to fibula 14 and the curved nail 120. The talus 16 is shown in both views and in relation to the true ankle joint 18 and the subtalar joint 20. Finally, in the lateral view, the calcaneus 22 is also depicted. The talus 16 is subjected to high stress in the section around the curved nail 120 insertion as expected. The calcaneus 22 shows stress concentration in the areas closer to the nail 120 since the curvature of the metal transfers the stresses to any area nearby.

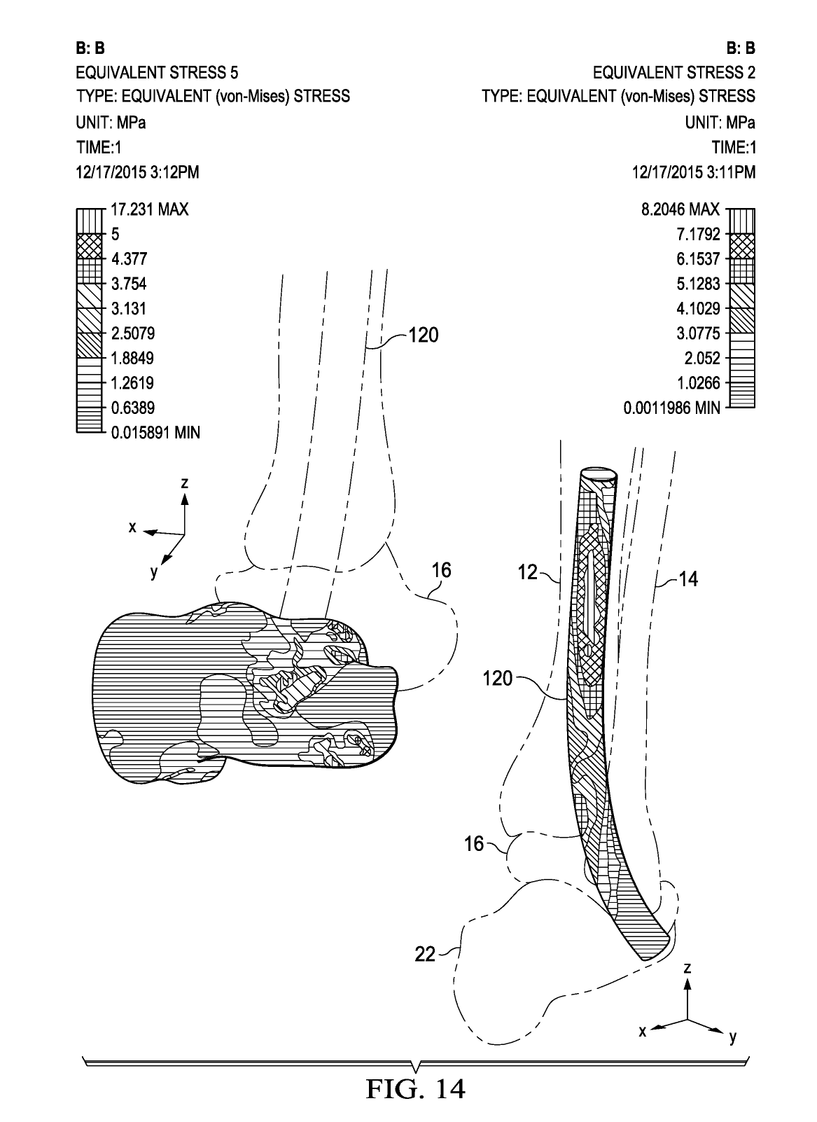

FIG. 14 shows a computational analysis in which the curved nail 120, tibia 12, and talus 16 are shown and in which the stress is concentrated in the upper section which carries the axial load at the first instance, it is expected that the addition of screws (not depicted) help to distribute the stresses in nail.

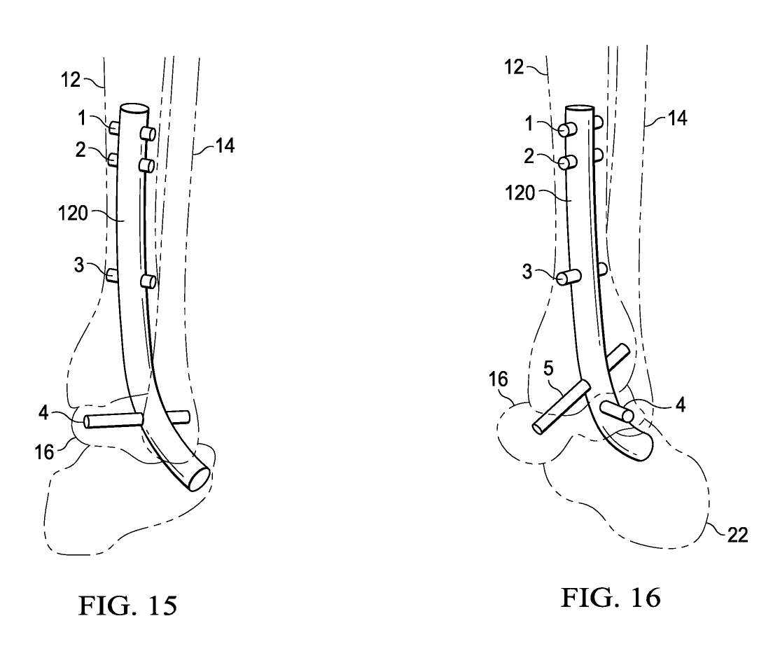

FIG. 15 shows the fixation of the nail to the bones with screws that ensures proper fixation and preventing movement during joint fusion and supporting and distributing stresses uniformly throughout the structure. The tibia 12 is shown in relation to fibula 14 and the curved nail 120. FIG. 15 shows screws 1, 2, 3, 4, is relation to the curved nail 120 of the present invention. The fixation of the nail to the bones with screws is very important and will perform different tasks such as ensuring proper fixation and preventing movement during joint fusion and supporting and distributing stresses uniformly throughout the structure. At least two screws are required to fix the nail to the tibia in the proximal section (1 & 2); the screws can be inserted from medial to lateral. In the distal section of the tibia another screw is required from medial to lateral (3). The talus will have to be fixed at least with a long screw inserted from posterior to anterior (4). In addition, a fifth inclined screw is recommended; it can be positioned from the tibia to the talus to increase fixation properties. In one non-limiting embodiment, with respect to the horizontal axis, the screws 1, 2 and 3 have no angle of inclination.

FIG. 16 shows the same image as in FIG. 15, but also illustrates screw 5. The tibia 12 is shown in relation to fibula 14 and the curved nail 120. Screw 5 is inserted in an inclined configuration, which is positioned from the tibia 12 to the talus 16 to increase fixation.

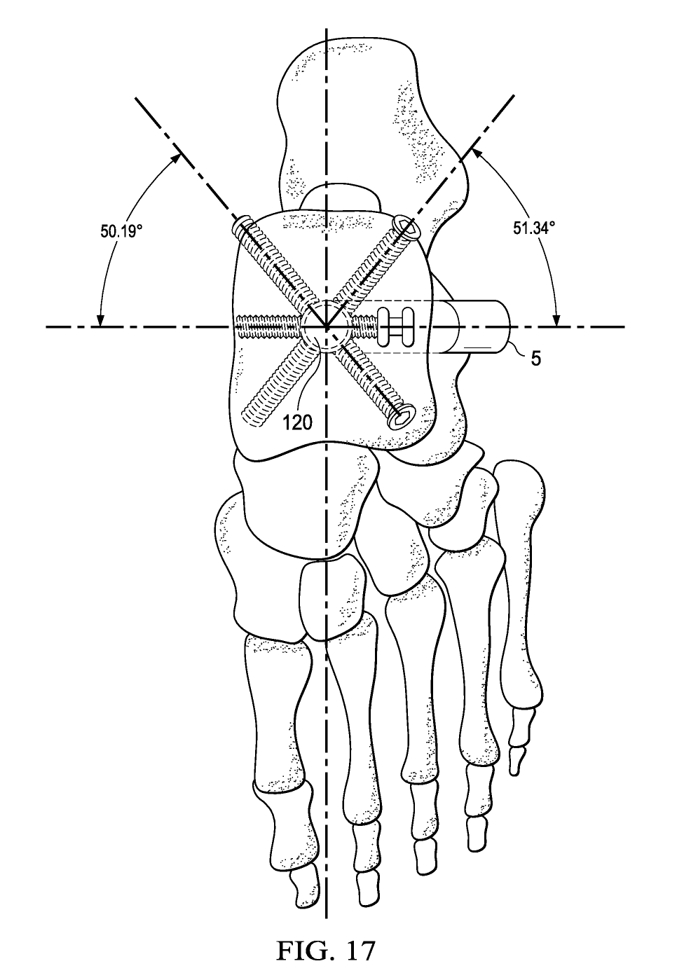

FIG. 17 a top view down the length of the curved screw 120 shows the screw 4 of the talus (4) and the screw 5 which crosses from tibia to talus (5) and the approximate angle of inclination.

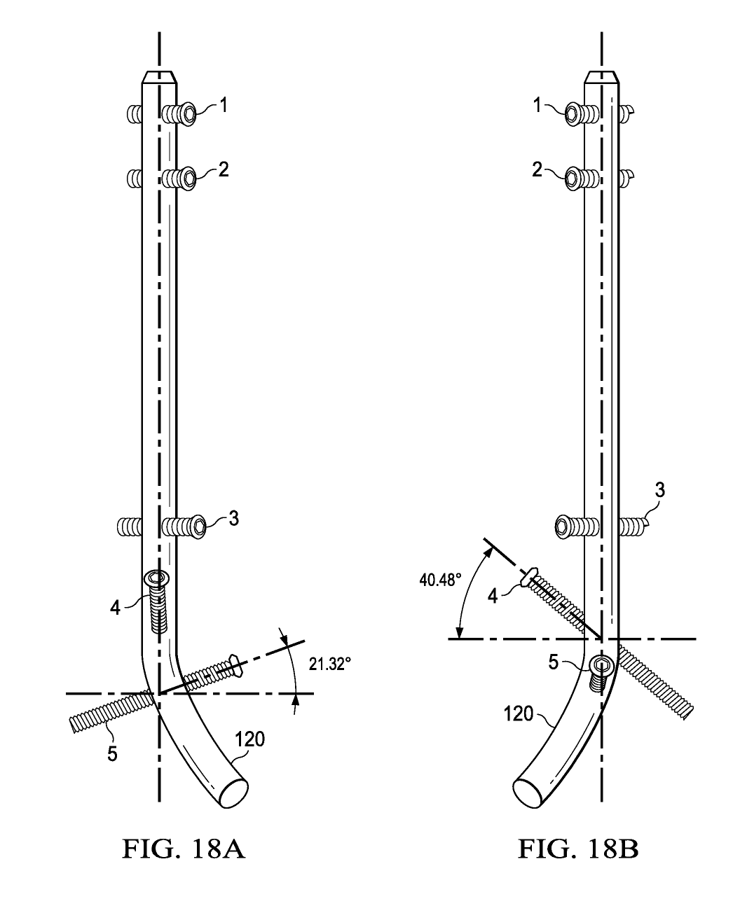

FIGS. 18A and 18B illustrate the angle of inclination for the curved nail 120, with talus screws 1-4 and for tibia/talus screw 5. Generally, the screws 1-5 are headed cortical-type screws with a screw diameter of 5.0 mm for all the bolts. Start-shape or hexagonal are good options for the screw head. One non-limiting material for the nail and the screws is a titanium alloy that has demonstrated lower mechanical failure rates and improved biocompatibility compare to stainless steel. The alloy Ti6AL4V ELI is one such option.

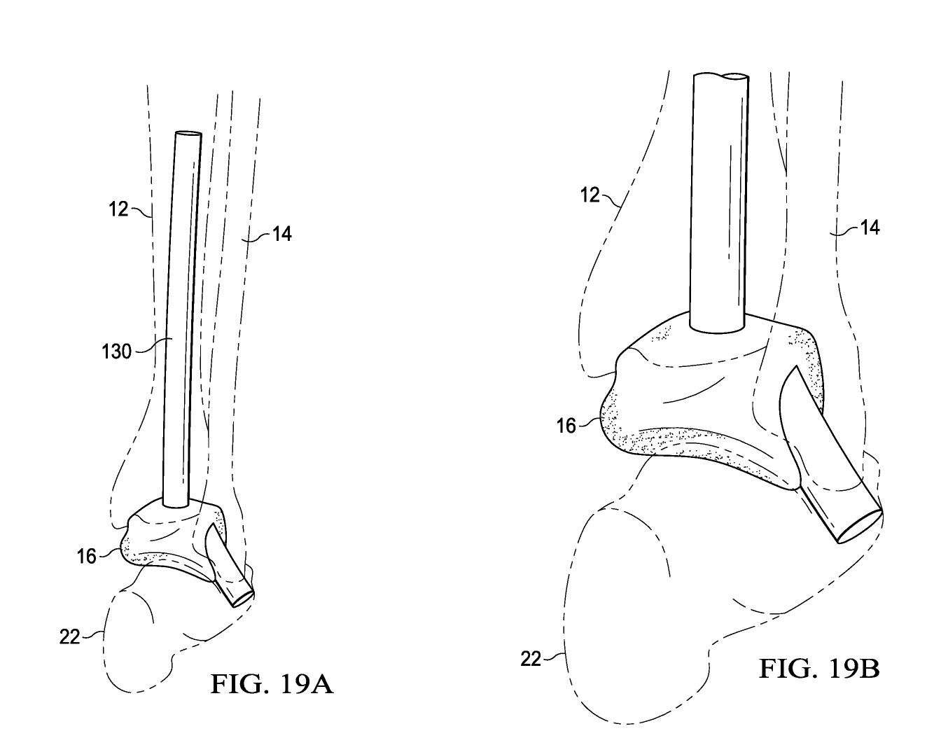

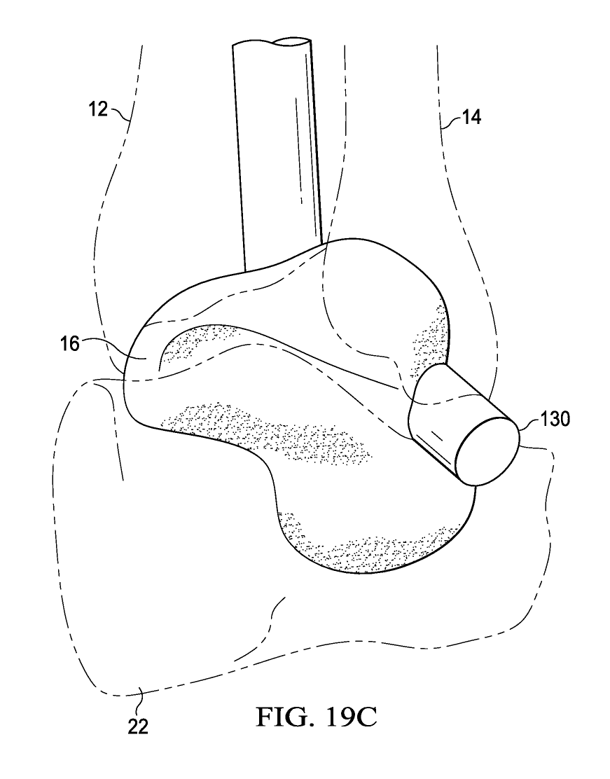

FIGS. 19A and 19B illustrate one option for the nail 130 of the present invention in which two straight segments connected each other; they have an angle of 37.24°. The tibia 12 is shown in relation to fibula 14 and the straight nail 130. The talus 16 is shown in both views in relation to the calcaneus 22, the tibia 12 and the fibula 14. This nail is called “straight nail” 130 and it is inserted from the talus 16 to the tibia 12.

FIG. 19C shows that the subtalar joint 20 is not affected when the nail 130 is inserted and positioned. The image below shows the clearance between the nail 130 and calcaneus 22 or between the nail 130 and subtalar joint 20. In this configuration, the nail 130 is not curved and the straight segments force an incline of the talus 16 with respect to the tibia 12 an angle of 37.24° when the nail 130 is being inserted. After the insertion is completed, the talus 16 and the tibia 12 can sustained their normal position without any issues, actually the straight segments of the nail 130 help to avoid movement between the two bones in one axis making easier the job of the securing screws. A curvature in the nail talus segment helps to have a deep insertion into the talus bone, so the designed can also be modified to have a curvature in the talus section but still a straight segment in the tibia 12.

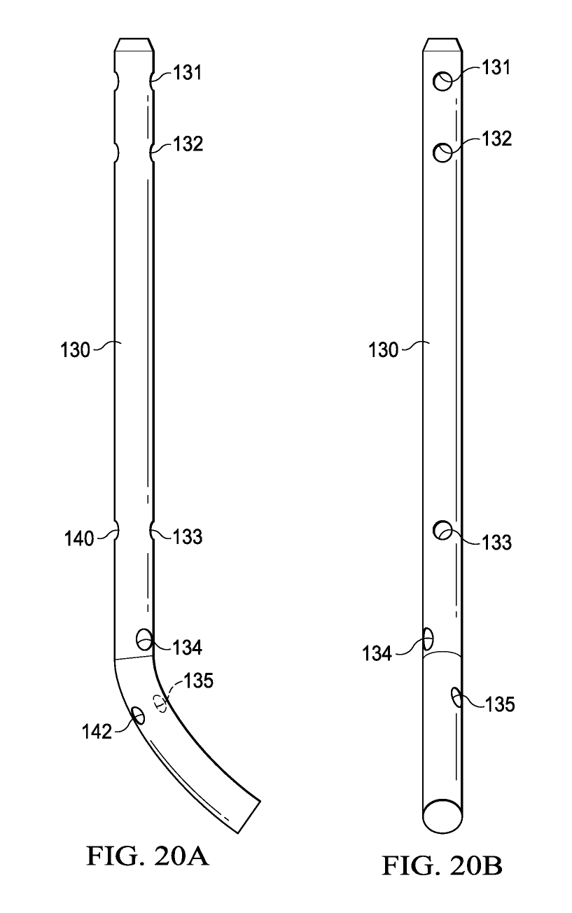

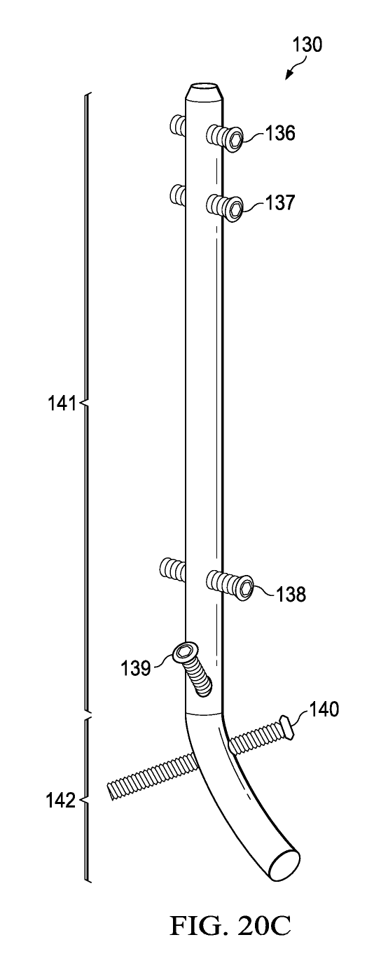

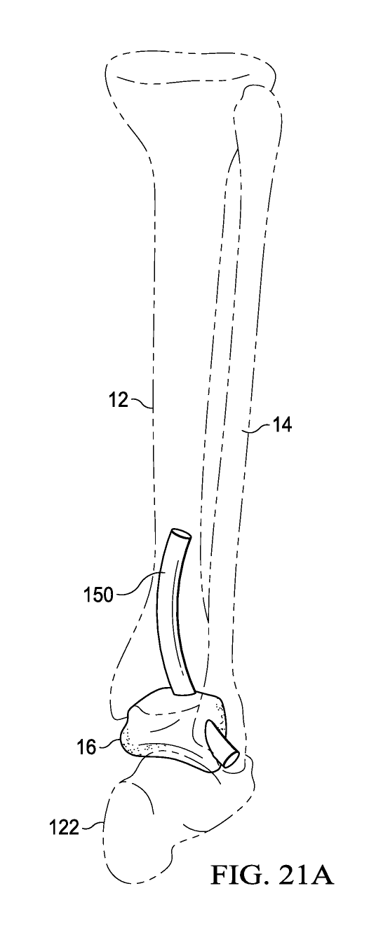

FIGS. 20A and 20B are a lateral and front view of the nail 130 and screw holes 131, 132, 133, 134 and 135. FIG. 20C is an isometric view of the nail 130, screws 136, 137, 138, 139, 140, in screw holes 11, 12, 13, 14 and 15. In certain non-limiting examples, the nail has one or more of the following features: Angle=37.24°, Radius of curvature R=7.72 cm, Nail length=22 cm (longer or shorter versions are possible in this design), Nail diameter=10 mm (8 mm version is also feasible), 5 screws: 5.0 mm diameter, 3 screws for the tibia, 1 for tibia/talus & 1 for the talus, and/or a talus rotation with respect to tibia is required (37.24°) when inserted the straight segment of the nail. The nail 130 has an upper portion that is a straight portion 141 and a lower portion that is a curved portion 142, which can be made in a single piece, or in two pieces, although certain advantages can be found in the screw being made from a single piece, that is, of unitary construction.

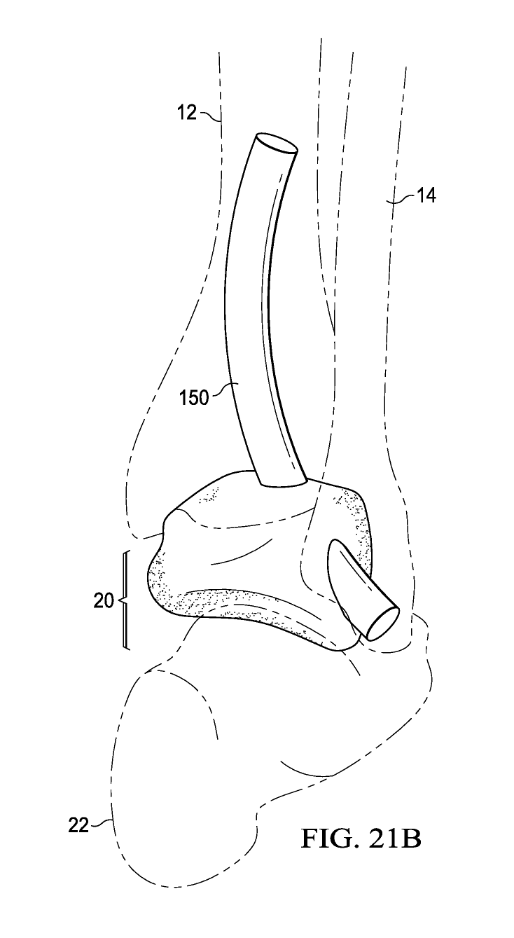

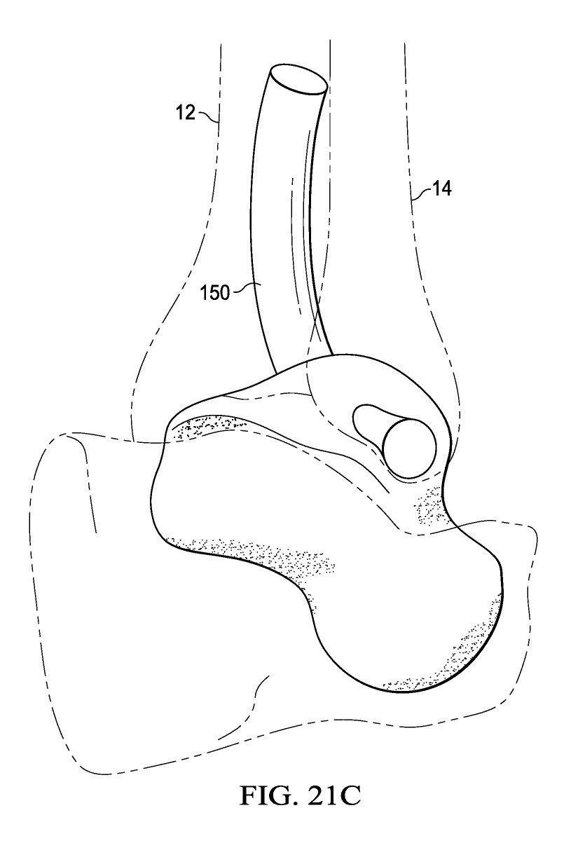

FIGS. 21A and 21B illustrate another option for the constant curved nail 150 of the present invention in which two straight segments connected each other and shows the clearance between the constant curved nail 150 and calcaneus 22 or between the nail 150 and subtalar joint 20, and is based on constant curved nail 150 geometry. The tibia 12 is shown in relation to fibula 14 and the straight nail 130. The talus 16 is shown in both views in relation to the calcaneus 22, the tibia 12 and the fibula 14. Only one curvature is designed and will pass through the tibia 12 and the talus 16. The constant curve nail 150 has a predominately single curvature.

FIG. 21C shows the clearance between the constant curved nail 150 trajectory and the subtalar joint 20, which prevents damage in the subtalar joint 20. In this embodiment, the constant curved nail 150 is not as close to the calcaneus 22 as in the figures above, however, the constant curved nail 150 is closer to the sidewall of the tibia. In terms of insertion of the nail into the bone, the advantage of this option is important because there is no need to move the talus with respect to the tibia when insert it. However, is it important to considerer that a curved hole is needed in talus section; if the bone drill is not curved then the advantage mentioned above may not be that important.

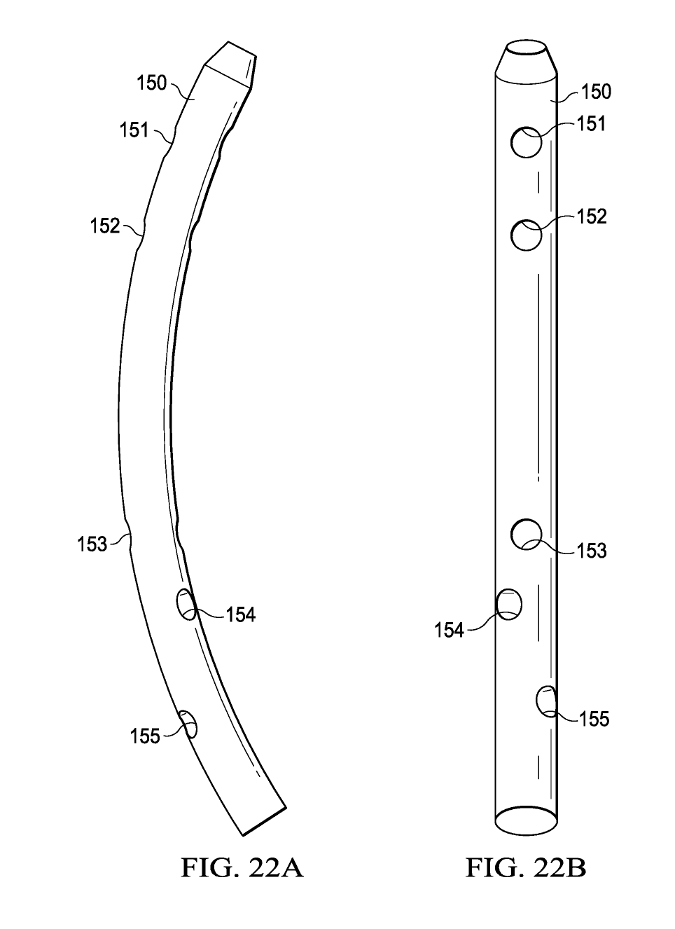

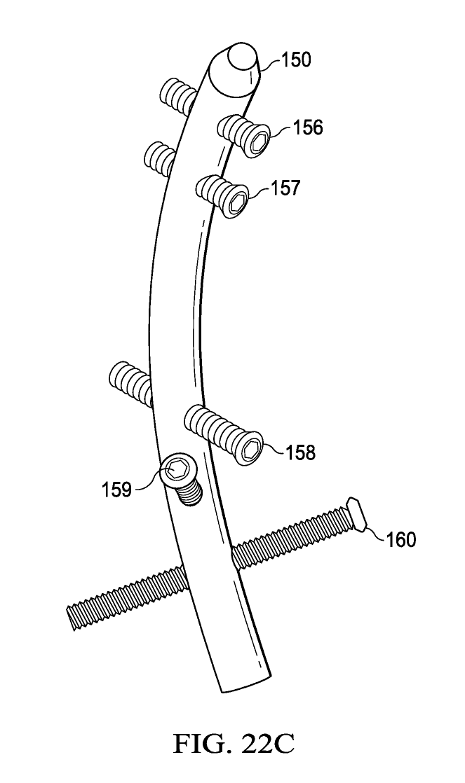

FIGS. 22A and 22B are a lateral and front view of the curved nail 150 and screw holes 151, 152, 153, 154 and 155. FIG. 22C is an isometric view of the curved nail 150, screws 156, 157, 158, 159, 160, in screw holes 151, 152, 153, 154 and 155. In certain non-limiting examples, the nail has one or more of the following features: Angle=16.58°, Radius of curvature R=12.79 cm, Nail length=13 cm (shorter version is feasible but difficult to increase length in this design), Nail diameter=10 mm (8 mm version is also feasible), 5 screws: 5.0 mm diameter, 3 screws for the tibia, 1 for tibia/talus & 1 for the talus.

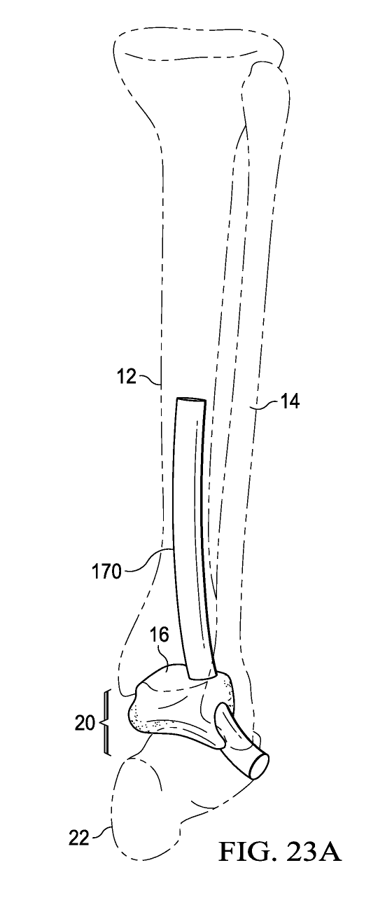

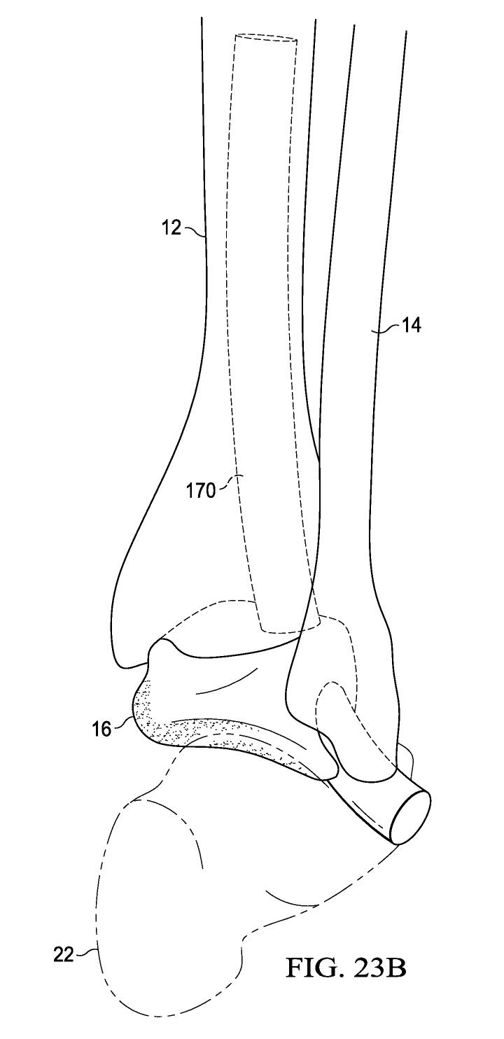

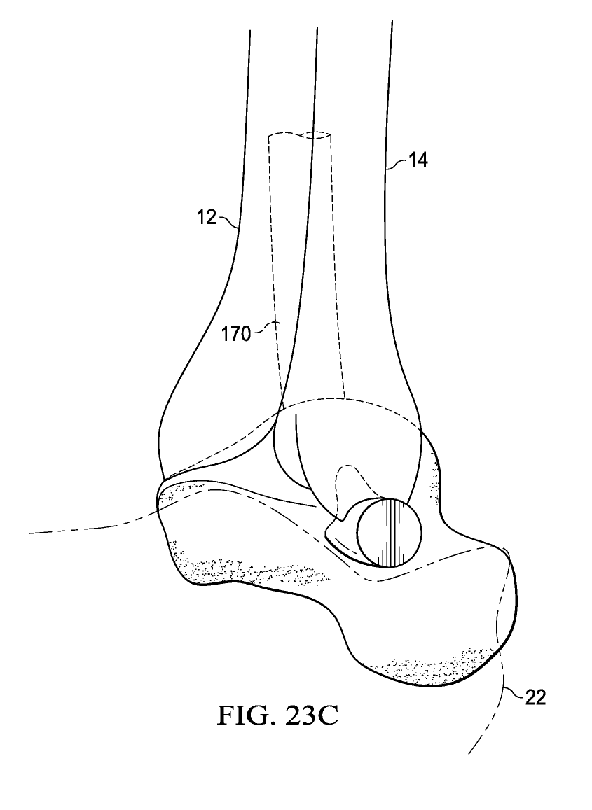

FIGS. 23A and 23B illustrate another option for the nail 170 of the present invention in which the curved nail has two different curves to form the nail 170. A first curvature is designed for the tibia segment (or upper segment) and a second more pronounce curvature is selected for the talus segment (or lower portion).

FIG. 23C shows the clearance between the nail 170 and subtalar joint is small but still enough to avoid damage in the calcaneus zone 22. This nail has the advantage of the curvature and very small movement is needed between the talus and the tibia when the nail is being implanted

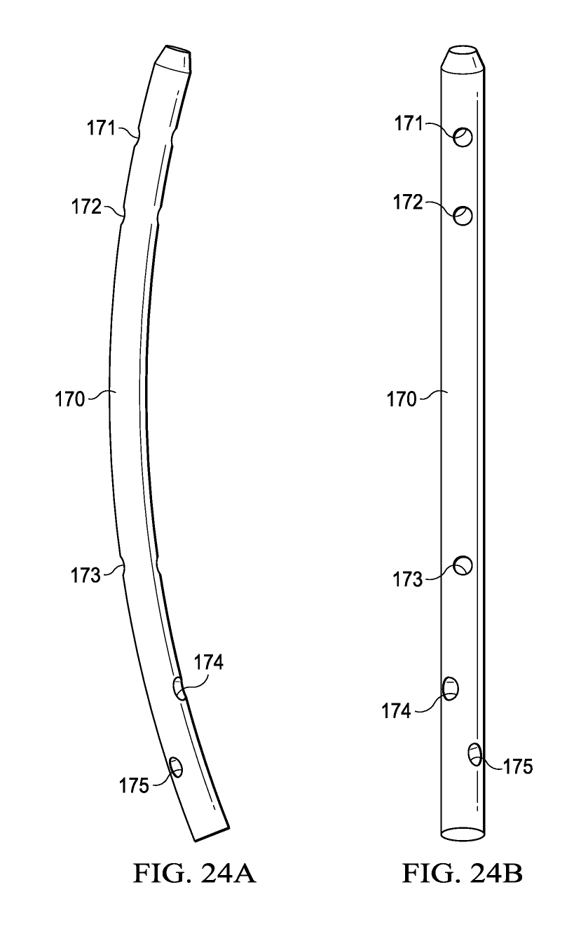

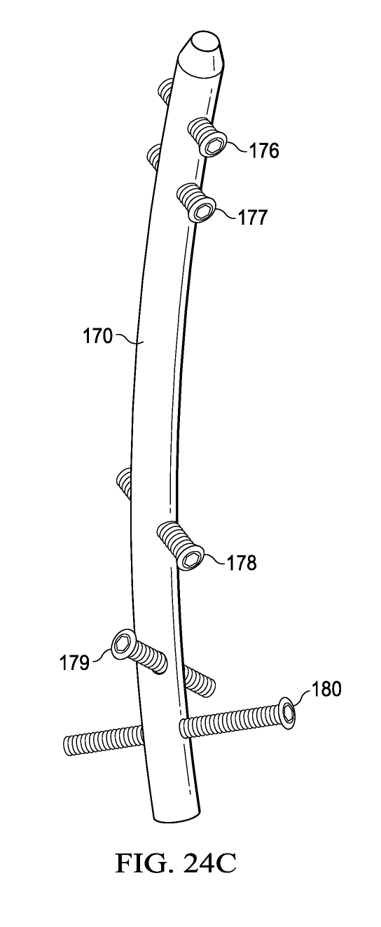

FIGS. 24A and 24B are a lateral and front view of the nail 170 and screw holes 171, 172, 173, 174 and 175. FIG. 24C is an isometric view of the nail 10, screws 176, 177, 178, 179, 180, in screw holes 171, 172, 173, 174 and 175. In certain non-limiting examples, the nail has one or more of the following features: Angle=10.99°, Radius of curvature R=32.07 cm, Nail length=20 cm (shorter version is feasible but difficult to increase length in this design), Nail diameter=10 mm (8 mm version is also feasible), 5 screws: 5.0 mm diameter, 3 screws for the tibia, 1 for tibia/talus & 1 for the talus.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A tibio-talar device for providing stabilizing support between a tibia and a talus comprising:
a bone nail adapted to traverse the tibia and the talus, wherein the bone nail is curved such that it traverses the tibia and enters the talus at an angle, wherein the nail is angled from lateral to medial and anterior to posterior, wherein the bone nail is configured to provide for intra medullary fusion of the ankle without obstructing the subtalar joint.

2. The device of claim 1, wherein the nail is further affixed to the tibia and talus with a biocompatible adhesive.

3. The device of claim 1, wherein the nail is contoured to lock proximally in the tibia from lateral to medial.

4. The device of claim 1, wherein the bone nail is provided in increments of approximately 5 cm.

5. The device of claim 1, wherein the bone nail is at least one of titanium or stainless steel.

6. The device of claim 1, wherein the bone nail has an upper and a lower portion, wherein the upper portion and the lower portion have the same curvature, the upper portion is straight and the lower portion is curved, or wherein the lower and upper portion are curved but have a radius of curvature.

7. The device of claim 1, wherein the bone nail has 2, 3, 4, 5, 6, or 8 openings that each are capable of supporting a screw, wherein the holes are positioned along the length of the bone nail, and wherein optionally one of the holes permits affixing the bone screw to the talus.

8. An ankle arthrodesis nail comprising:
a bone nail adapted to traverse the tibia and the talus, wherein the bone nail is curved such that it traverses the tibia and enters the talus at an angle, wherein the nail is angled from lateral to medial and anterior to posterior, and wherein the bone nail is configured to provide for intra medullary fusion of the ankle, wherein the nail locks proximally in the tibia from lateral to medial, without obstructing the subtalar joint.

9. The device of claim 8, wherein the nail is further affixed to the tibia and talus with a biocompatible adhesive.

10. The device of claim 8, wherein the nail is contoured to lock proximally in the tibia from lateral to medial.

11. The device of claim 8, wherein the bone nail is provided in increments of approximately 5 cm.

12. The device of claim 8, wherein the bone nail is at least one of titanium or stainless steel.

13. The device of claim 8, further comprising one or more opening to receive screws that pass from the tibia or the fibula through the nail to the talus.

14. The device of claim 8, wherein the bone nail has an upper and a lower portion, wherein the upper portion and the lower portion have the same curvature, the upper portion is straight and the lower portion is curved, or wherein the lower and upper portion are curved but have a radius of curvature.

15. The device of claim 8, wherein the bone nail has 2, 3, 4, 5, 6, or 8 openings that each are capable of supporting a screw, wherein the holes are positioned along the length of the bone nail, and wherein optionally one of the holes permits affixing the bone screw to the talus.

16. A method for conducting an ankle arthrodesis system for providing stabilizing support between a tibia and a talus, the system comprising:
identifying a patient in need of an ankle arthrodesis;
a bone nail adapted to traverse the tibia and the talus, wherein the bone nail is curved such that it traverses the tibia and enters the talus at an angle, wherein the nail is angled from lateral to medial and anterior to posterior, wherein the bone nail is configured to provide for intra medullary fusion of the ankle; and
anchoring the bone nail without obstructing the subtalar joint.

17. The method of claim 16, wherein opening is defined further as being adapted for the bone anchor to at least one of traverse from talus to tibia or traverse from tibia to talus.

18. The method of claim 16, wherein the nail is further affixed to the tibia and talus with a biocompatible adhesive.

19. The method of claim 16, wherein the nail is contoured to lock proximally in the tibia from lateral to medial.

20. The method of claim 16, wherein the bone nail is provided in increments of approximately 5 cm.

21. The method of claim 16, wherein the bone nail is at least one of titanium or stainless steel.

22. The method of claim 16, further comprising inserting one or more screws through one or more openings, wherein the screws pass from the tibia or the fibula through the nail to the talus.

23. The method of claim 16, wherein the bone nail has an upper and a lower portion, wherein the upper portion and the lower portion have the same curvature, the upper portion is straight and the lower portion is curved, or wherein the lower and upper portion are curved but have a radius of curvature.

24. The method of claim 16, wherein the bone nail has 2, 3, 4, 5, 6, or 8 openings that each are capable of supporting a screw, wherein the holes are positioned along the length of the bone nail, and wherein optionally one of the holes permits affixing the bone screw to the talus.
